(19) United States
(12) Plant Patent Application Publication
(10) Pub. No.: US 2003/0061646 P1
(43) Pub. Date: Mar. 27, 2003

(54) Clematis plant named 'Sprinkles'

(76) Inventor: James P. VanLaeken, Walworth, NY (US)

Correspondence Address: Fred L. Denson, 14 East Main Street, P.O. Box 801, Webster, NY, 14580, US

(21) Appl. No.: 09/964,948

(22) Filed: Sep. 27, 2001

(51) Int. Cl. A01H 5/00

(52) U.S. Cl. PLT/228

(57) ABSTRACT

A new variety of clematis plant named ‘Sprinkles’. The variety is characterized by wavy edged petals having sprinkles of white distributed throughout the petal surface and a white bar that extends along the center longitudinal axis of each petal.

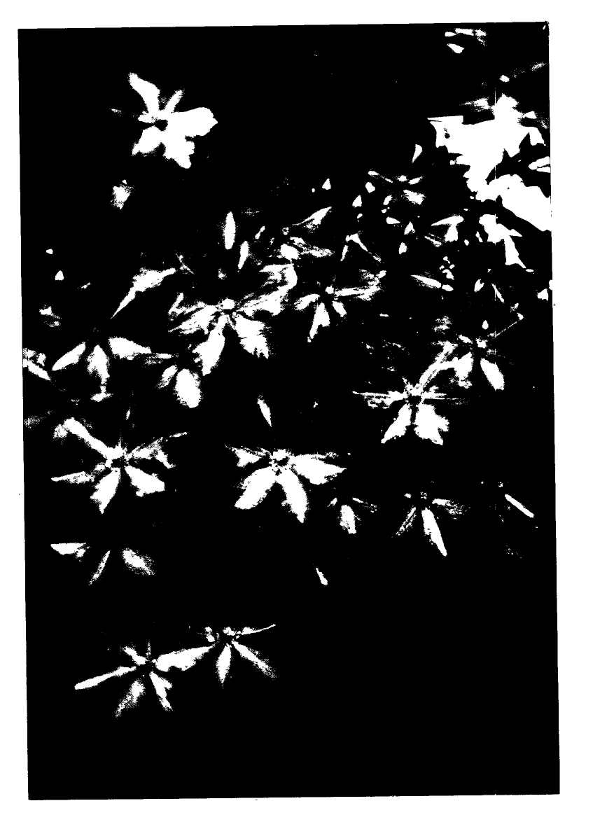

### BACKGROUND OF THE NEW VARIETY

The present invention relates to a new and distinct variety of clematis plant known by the cultivar name ‘Sprinkles’.

The new variety was discovered in a controlled planting of cultivated plants in a clematis nursery in Walworth, N.Y. in 1994. Plants were asexually propagated and observed over the next four years. In 1998, an offspring was selected and planted in an outside garden area and observed over the next two years. Asexual propagation by vegetative cuttings has shown that the propagules are identical to the original plant in all distinguishing characteristics. The stability of the variety is evidenced by the distinguishing characteristics remaining true to form from generation to generation. The parentage of the variety is unknown.

### SUMMARY OF THE INVENTION

Among the features which distinguish the new variety from other presently available and known commercial clematis cultivars are the unique color of the flower and the long blooming habit of the plant. The flower has sprinkles of white which are distributed throughout the petals. A petal typically carries a white bar that extends along its center longitudinal axis. While the white bar is not dominant or very pronounced or consistent throughout the flowers, it is consistent throughout the plarLt itself. Their wavy edged petals and rose-tipped stamens further distinguish the flowers.

### RELEVANT PRIOR ART

The new variety is somewhat similar in appearance to the Ernest Markham variety as shown in the Spring Valley Greenhouse, Inc. catalogue (4^{th} Edition) at page 9. While the flowers of both varieties are about the same size and have wavy edged petals, there is a marked difference in the coloration of the petals. The petals of the Ernest Markham variety are a solid, magenta-red (Pantone®#512C), whereas the petals of the new variety have edges which are rose pink (Pantone®#246U) and which fade to white in the center thereby forming a white bar in the middle of the petal. Moreover, white is also sprinkled throughout the petals creating an aesthetically soft appearance.

### BRIEF DESCRIPTION OF THE PHOTOGRAPH

The accompanying photograph illustrates the overall appearance of the new variety. The flower and foliage colors as shown are, within the limitations of color photography, accurate and true reproductions of the actual colors of the new variety. The photographic view is a front elevation of typical Clematis Sprinkles in full bloom.

### BOTANICAL DESCRIPTION

The following information is based on plants grown in Walworth, N.Y. Color references are based on using the Pantone® matching system.

I. Plant

A. Form: upright climbing or spreading

B. Growth: vigorous, vining

1. Height.—approximately 10 feet to 12 feet

C. Foliage: deciduous, compound-trifoliate, opposite

1. Size.—medium

2. Quantity.—average

3. Color.—dark green (Pantonet#371C)

4. Shape.—lower leaves sometimes simple; normally three ovate leaflets in pinnate form.

5. Texture.—upperside-glabrous veined, underside-ribbed

6. Petiole color.—green/red

7. Internodes.—cylindrical, approximately from about 5 inches to about 8 inches

8. Disease .—a. subject to any disease that normally affects clematis b. has shown resistance to powdery mildew

D. Wood

1. New shoots.—a. Color: light green, turning deep red/purple b. Bark: smooth

2. Old Wood.—a. Color: light brown b. Bark: rough

E. Hardiness: Zones 4 through 8

II. Flower

A. Blooming habit: recurrent, June to September based on climate in Rochester, N.Y. area

B. Bud:

1. Size.—from about 1.5 inches to about 2.0 inches,

2. Form.—elliptic to ovate

3. Color.—green/grayish white

C. Stem:

1. Length.—from about 6 inches to about 8 inches

2. Aspect.—vertical

3. Strength.—erect

4. Color.—green, turning red/purple with age

D. Bloom

1. Size.—from about 4 inches to about 6 inches

2. Form.—l round, very symmetrical with six (6) petals of equal size overlapping with wavy margins

3. Color.—edges of petals are rose pink (Pantones #246U) fading to white in the center; white is also sprinkled throughout the petals giving the plant a very soft look to the human eye

4. Characteristics.—borne on 6 inch to 8 inch stems; suitable for cut flower arrangements

5. Quantity.—very floriferous, resulting from multiple buds formed on each stem

E. Petals

1. Form.—obovate

2. Arrangement.—regularly arranged

3. Fragrance.—none

4. Color.—(see II D 3 above)

III. Reproductive Organs

A. Stamens, filaments and anthers

1. Arrangement.—regularly arranged

2. Color.—a. Stamens: cream colored with rose tips to match outer edges of petals

B. Pollen

1. Color.—off white

C. Styles: bunched, even, average length

D. Stigmas:

1. Color.—rose-white

I claim:

1. A new and distinct variety of clematis plant named ‘Sprinikles’ as shown and described herein.
